(19) United States
(12) Patent Application Publication
(10) Pub. No.: US 2007/0015113 A1
(43) Pub. Date: Jan. 18, 2007

(54) Device for separating between the upper and lower jaws and method using the same

(76) Inventors: Eran Lavi, Givatayim (IL); David Madjar, Ramat Gan (IL); Moshe Evron, Hod Hasharon (IL)

Correspondence Address: KENYON & KENYON LLP, ONE BROADWAY, NEW YORK, NY, 10004, US

(21) Appl. No.: 10/539,332

(22) PCT Filed: Dec. 11, 2003
(86) PCT No.: PCT/IL03/01063

§ 371 Date: May 1, 2006

(30) Foreign Application Priority Data
Dec. 15, 2002 (IL) 153451

(51) Int. Cl. A61C 5/00

(52) U.S. Cl. 433/217.1

(57) ABSTRACT

A device for separating between the upper and lower jaws is disclosed. The device comprises at least one substantially U-shaped rib and a deformable member associated therewith such that said member gradually changes its shape when a pressure is applied on the at least one rib by the jaws. The device is suitable for preventing self-inflicting or externally inflicted injury or for suppressing upper airway resistance syndrome, sleep apnea syndrome or snoring. By filling at least one reservoir formed in the deformable member with a beneficial or edible material and inserting the device into the oral cavity of a subject such that said deformable member separates an upper set of posterior teeth from a corresponding lower set of posterior teeth, the beneficial or edible material may be delivered to the subject.

### FIELD OF THE INVENTION

The present invention relates to the field of protection devices. More particularly, the invention relates to a device which prevents a self-inflicting or externally inflicted injury, e.g. during an epileptic seizure or during a sport related activity, with the use of two pliable support units for separating corresponding upper and lower posterior teeth and for retaining each set of posterior teeth within a fixed boundary, and therefore the tongue or inner cheeks of a subject are not in danger of being lacerated.

### BACKGROUND OF THE INVENTION

Epileptic seizures result from a transient excessive discharge of the electrical activity of cerebral neurons characterized by sudden, brief attacks of altered consciousness, motor activity, sensory phenomena, or inappropriate behavior. Many types of epileptic seizures occur, and most are classified within two main categories: partial seizures, when the excessive electrical activity is limited to one area in the brain, and generalized seizures, when the excessive electrical activity encompasses the entire brain.

There are two types of partial seizures: simple and complex. Consciousness is not impaired when an individual experiences a simple partial seizure. The affected individual may have tingling sensations, abnormal motor movement, hear a buzzing sound, feel unexplained fear, have auditory, visual, and/or olfactory hallucinations. Complex partial seizures usually involve one lobe of the brain and can result in uncontrolled movements, impaired consciousness and/or automatic actions such as uncontrollable chewing.

Within the generalized category there are two main types of seizures: tonic-clonic and absence. Absence seizures may have basically similar symptoms as those associated with complex partial seizures; however, the entire brain is involved in the abnormal electrical activity.

Complex partial or generalized seizures are immediately preceded by an aura, which is a sensory or psychic manifestation that represents a seizure onset.

When a generalized tonic-clonic seizure occurs the muscles suddenly contract causing the patient to fall and lie rigidly on the ground for ten to thirty seconds. A high pitched sound from the throat may occur along with a possible loss of bowels and/or loss of bladder control. The body trembles as the muscles alternate between a relaxed and rigid state. A seizure usually lasts for about two to three minutes. After the occurrence, an individual suffering from such a seizure usually awakes in a state of confusion and extreme fatigue. Bystanders cannot stop the seizure, and can only prevent serious injury by placing the subject on his side so as to prevent him from choking on his own vomit.

As previously mentioned, a subject during complex partial or generalized tonic-clonic seizures is usually characterized by uncontrollable bodily movement. Such uncontrollable bodily movement is liable to result in self-inflicting injury, which at times is irreversible. For example, uncontrolled biting is liable to result in the severing of the tongue or of a portion of the inner cheek. An apparatus is therefore needed for the prevention of tongue or inner cheek damage during an epileptic seizure.

U.S. Pat. No. 4,041,937 discloses a medical implement in the form of a planar blade body for compressing and holding a patient's tongue. The implement includes a bite guard positioned on the blade body so as to be in registration with confronting upper and lower front teeth of a patient upon insertion of the blade body within the mouth of the patient. Although this implement prevents damage to the tongue and to the front teeth during a seizure, it nevertheless does not prevent injury to the tongue, the inner cheek, or to the molars resulting from a biting motion which is not solely up and down, but also is transversal. Also, due to the relatively small minimal separation between the upper and lower front teeth, approximately equal to the thickness of the bite guard, the patient cannot release vomit from the oral cavity and is liable to choke if the vomit is swallowed. Additionally, the medical implement can be ejected from the mouth of the patient during a convulsion of the tongue when the plane body is not depressed by a medical assistant.

U.S. Pat. Nos. 4,179,815, 5,469,865, 6,241,518, 6,244,866 and 6,241,521 disclose different types of dental appliances for maintaining the mouth of a patient in an open position during a dental procedure, and suffer from at least one of the aforementioned drawbacks. Also, a subject who sensed an aura may not be able to easily and speedily insert any of these dental appliances within his mouth before the onset of the imminent epileptic seizure. Furthermore these dental appliances are intended for a dental application during which the masticatory muscles of the patient are relaxed; however, the interjaw compressive forces during an epileptic seizure are liable to result in an excessive stress concentration and eventual deformation or even failure of a dental appliance.

U.S. Pat. No. 5,386,821 discloses a U-shaped bite-block for endotracheally intubated patients made of a hard but pliable material, such that a curved rib connects the two legs of the bite-block. The bite-block is dimensioned to be held in place by the compressional contact of the molars of the patient. The disadvantage of this arrangement for use during an epileptic seizure when jaw movement is uncontrollable is that the bite-block may become dislodged from between the teeth when the jaws are opened to their fullest extent, such as during vomiting or yawning, and therefore may not be able to separate the upper and lower molars when the jaws return to their original position.

An oral device with an anterior opening, which was fabricated from copolyester foil and autopolymerizing resin for the treatment of upper airway resistance syndrome by moving the position of the mandible and tongue forward in order to minimize the possibility of oropharyngeal obstruction, is disclosed by Kazuya Yoshida, “Oral Device Therapy for the Upper Airway Resistance Syndrome Patient,” The Journal of Prosthetic Dentistry, Vol. 87, No. 4. April, 2002, pp. 427-429. The illustrated oral device is adapted to separate the upper and lower jaws at an intermediate jaw angular opening, and may become dislodged from between the teeth when the jaws are opened to their fullest extent. Also, the oral device is uniformly stiff and does not allow for jaw closure when a subject is asleep, resulting in discomfiture and in drying of the mouth. If the material of the oral device were less stiff, the jaws could not be urged to be opened to their fullest extent.

It is an object of the present invention to provide a protection device against self-inflicting injury during an epileptic seizure.

It is an additional object of the present invention to provide a protection device which prevents injury to the tongue, inner cheeks and teeth of a subject during an epileptic seizure.

It is an additional object of the present invention to provide a protection device which allows for the release of vomit from the oral cavity.

It is an additional object of the present invention to provide a protection device which follows the movement of the jaws.

It is an additional object of the present invention to provide a protection device that cannot be ejected from the oral cavity as a result of a convulsion or a movement of the tongue.

It is another object of the present invention to provide a protection device which is easily and speedily insertable by a subject within his mouth before the onset of an imminent epileptic seizure.

It is another object of the present invention to provide a protection device which continues to remain between a set of upper and lower teeth despite an opening of the jaws less than or equal to their greatest extent.

It is another object of the present invention to provide a protection device which is configured to bear interjaw compressive forces.

It is yet another object of the present invention to provide a device which can deliver a pharmaceutically active agent to a subject during an epileptic seizure or during other occurrences of impaired consciousness, or for events that require a slow release of a pharmaceutically active agent.

Other objects and advantages of the invention will become apparent as the description proceeds.

### SUMMARY OF THE INVENTION

The present invention provides a device for separating between the upper and lower jaws, comprising at least one substantially U-shaped rib and a deformable member associated therewith such that said member gradually changes its shape when a pressure is applied on said at least one rib by the jaws.

In one aspect, the device is a protection device which prevents a self-inflicting or externally inflicted injury.

In another aspect, the device is a device for suppressing upper airway resistance syndrome, sleep apnea syndrome or snoring.

The device preferably comprises two substantially U-shaped ribs, connected at at least one end to one another, and a deformable member positioned between them such that said member gradually changes its shape when a pressure is applied on said ribs by the jaws.

In a preferred embodiment of the present invention, the device comprises:
a) two U-shaped ribs having a lingual and buccal side, each of said ribs having a curvature corresponding to the curvature of a dental arch and subtending a majority of its length;
b) two support units attached at each posterior end of a pair of said ribs on the lingual side thereof, such that said two support units have bilateral symmetry about a plane coincidental with an anteriorly disposed central portion of each of said ribs and that each of said ribs is separated one from the other, at a given point along a rib, by a separation substantially corresponding to an essentially maximum jaw angular opening when said support units are not compressed; and
c) an upper and a lower tooth receiving means for each support unit, each of said tooth receiving means extending the entire length of a corresponding support unit and adapted to retain posterior teeth therein, borders of each of said tooth receiving means being defined by a wall of a support unit longitudinally protruding from a lingual side of a corresponding tooth receiving means and a portion of a rib longitudinally protruding from the buccal side thereof

Preferably, each support unit is configured in such a way and produced from a suitable material so as to follow the movement of the jaws.

Preferably, each support unit comprises a plurality of longitudinally disposed fins which are compressible upon application of interjaw forces and which can return to their original dimensions following the relaxation of the jaw muscles.

As referred to herein, “longitudinal” is in a direction similar to, but not identical to, to the disposition of the teeth. “Buccal” is in a direction towards the cheeks while “lingual” is in a direction towards the tongue. “Inwards” is in a direction towards the oral cavity, away from the teeth.

In one aspect, each support unit is provided with solid lingual walls.

In one aspect, each support unit is enclosed by solid lingual and buccal walls, a plurality of chambers being defined by said solid walls and by two adjacent fins for the insertion therein of therapeutic material.

In one aspect, each fin comprises a first portion proximate to a first rib, a second portion proximate to a second rib, and a central arcuate portion connecting said first portion and said second portion, said first portion and second portions being symmetrical about a plane which passes through a junction connecting said first and second ribs and which separates a support unit into two separate sections.

The first and second portions are preferably planar elements, all first portions of a given support unit being mutually parallel and all second portions of a given support unit being mutually parallel.

The length of each first and second portion preferably progressively decreases from the most anteriorly disposed fin to the most posteriorly disposed fin whereby the first rib is inclined at a predetermined angle with respect to the second rib.

This predetermined angle preferably corresponds to the essentially maximum jaw angular opening, which allows for temporary deformation of a support unit during an uncontrolled biting motion without any risk of being dislodged from between a corresponding set of upper and lower posterior teeth as the jaws are separated to their fullest extent.

In one aspect, the tooth receiving means is a planar surface which abuts the same longitudinal end of each fin of a support unit.

In another aspect, the tooth receiving means is the plurality of chambers. In another preferred embodiment of the present invention the device further comprises at least one reservoir suitable for housing a beneficial or edible material.

Said beneficial or edible material is delivered to a subject by means selected from the group of the pressure applied to the deformable member by the jaws, temperature activated means, moisture activated means, timed release means and control means. The control means is preferably at least one electronic component and circuitry.

The at least one reservoir is additionally suitable for housing a measuring or control means. In one aspect, the measuring or control means measures or controls electric current and/or saliva secretion.

In one aspect, the beneficial or edible material is essentially immediately deliverable upon activation of the delivery means.

The beneficial or edible material is preferably a pharmaceutically active agent.

In one aspect the beneficial or edible material is a scent or taste additive to the oral cavity.

The beneficial or edible material is in the form selected from the group of liquid, aerosol, powder, gas, and encapsulated form.

In one aspect, each reservoir is a recess formed in the deformable member.

The central anteriorly disposed portion of each rib is preferably placeable on a corresponding gum when the device is inserted within the oral cavity.

The central anteriorly disposed portion of each rib is preferably an arcuate member which is shaped so as to prevent damage to the frenulum of a corresponding lip.

The device preferably further comprises a handle integrally formed with a rib, longitudinally inwards from a corresponding central anteriorly disposed portion.

The device is formed with a centrally and anteriorly located airway for the release of vomit from the oral cavity. The airway is formed between two opposing ribs and two opposing deformable members.

In one aspect, the at least one rib and the deformable member are produced from different materials, the yield strength of the at least one rib being significantly greater than that of the deformable member.

In another aspect, the at least one rib and the deformable member are produced from the same material.

In one aspect, the material of the deformable member hardens after use.

In one aspect, the device is disposable and for one-time use.

In another aspect, the device is reusable.

In one aspect, the device is packageable in a container which breaks upon removal of the device from said container. The container preferably is provided with a retaining means for retaining the container in an accessible location.

The present invention also provides a method for delivering material to a subject, comprising:

a) providing a device with two substantially U-shaped ribs, connected at at least one end to one another, and a deformable member positioned between them formed with at least one reservoir suitable for housing a beneficial or edible material;

b) filling each of said at least one reservoir with a beneficial or edible material;

c) inserting said device into the oral cavity of said subject such that said deformable member separates an upper set of posterior teeth from a corresponding lower set of posterior teeth; and

d) allowing said beneficial or edible material to be delivered to said subject.

In one aspect, the beneficial or edible material is delivered to the subject upon application of pressure onto said deformable member by the jaws.

In another aspect, the beneficial or edible material is delivered to the subject when the temperature within the oral cavity of the subject is greater than a predetermined value.

In another aspect, the beneficial or edible material is delivered to the subject when the moisture level within the oral cavity of the subject is greater than a predetermined value.

In another aspect, the beneficial or edible material is delivered to the subject after a predetermined time following insertion of the device within the oral cavity.

In another aspect, the beneficial or edible material is delivered to the subject upon activation of control circuitry.

### BRIEF DESCRIPTION OF THE DRAWINGS

In the drawings:

FIG. 1 is a perspective view of a protection device according to one preferred embodiment of the present invention;

FIG. 2 is a front view of the protection device of FIG. 1;

FIG. 3 is a side view of the protection device of FIG. 1;

FIG. 4 is a perspective view of the protection device of FIG. 1, showing a teeth retention surface, while FIG. 4A shows a different type of teeth retention surface and FIG. 4B shows a different fin configuration;

FIG. 5 is a perspective view of a protection device according to a second preferred embodiment of the present invention;

FIG. 6 is a perspective view of a protection device according to a third embodiment of the present invention;

FIG. 7 is a perspective view of a container for holding the protection device of FIG. 1; and

FIG. 8 illustrates the removal of a protection device from its container.

### DETAILED DESCRIPTION OF PREFERRED EMBODIMENTS

The protection device of the present invention incorporates two pliable support units which follow the movement of the jaws and are adapted for separating corresponding upper and lower posterior teeth and for retaining each set of posterior teeth within a fixed boundary during an epileptic seizure, and therefore the tongue or inner cheeks of a subject are not in danger of being lacerated. Accordingly, the tongue of the subject is not restrained and is free to facilitate the ejection of vomit from the oral cavity as well as the swallowing of saliva or other liquids. The subject may advantageously insert the protection device within his mouth during the manifestation of an aura, immediately preceding an epileptic seizure.

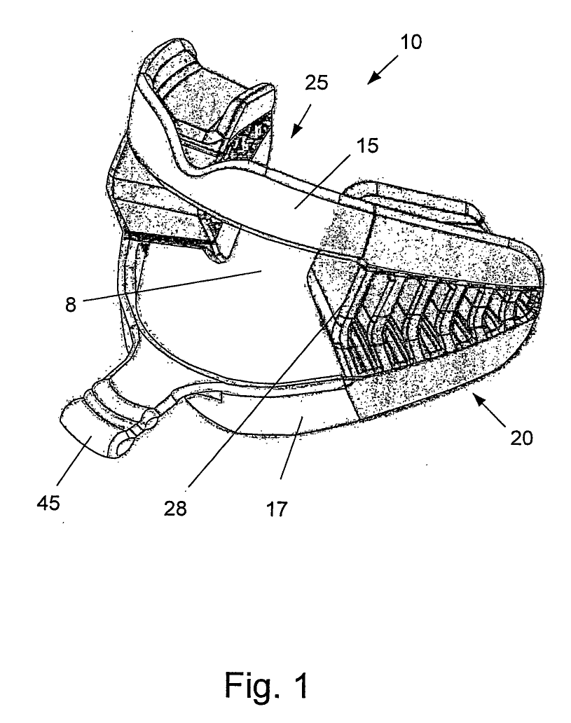

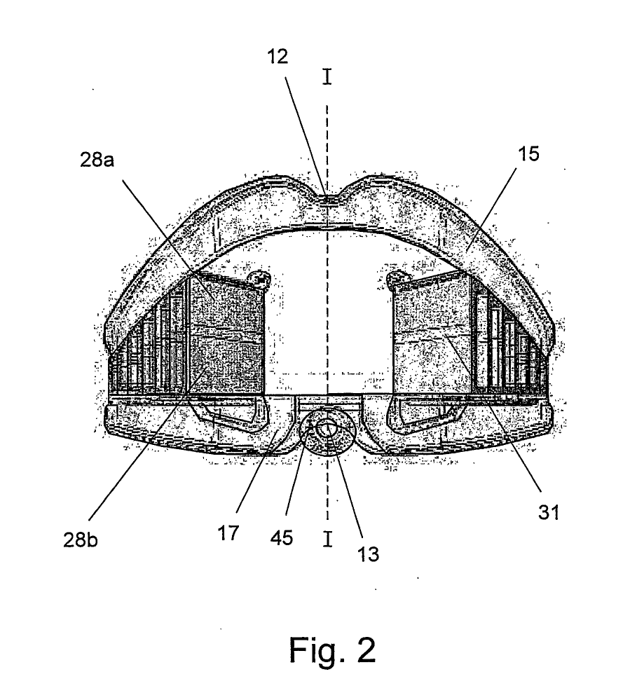

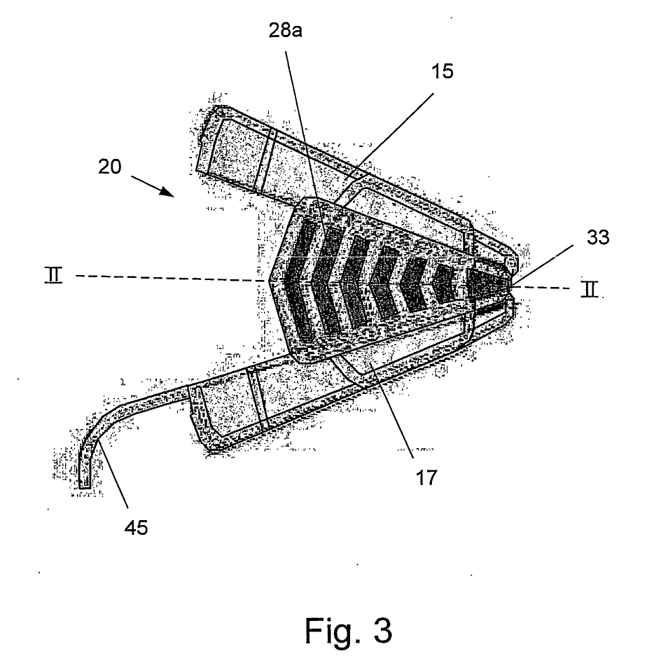

Referring now to FIG. 1, one preferred embodiment of the protection device of the present invention is illustrated and is indicated generally by numeral 10. Protection device 10 has U-shaped ribs 15 and 17, wherein each rib has a curvature corresponding to that of a dental arch, subtending a majority of its length when inserted within the mouth of a subject. The relative position of ribs 15 and 17 is determined by support units 20 and 25, each of which connects the two ribs at a different posterior end, on the lingual side thereof, so that the two ribs assume the general shape of the jaws such that the separation between anterior teeth is greater than the separation between posterior teeth. Ribs 15 and 17 are connected, e.g. by bonding or by fusing, to form a substantially longitudinally disposed junction 33 (FIG. 3) at each posterior end of the protection device. When the support units are not deformed, the separation between ribs 15 and 17 corresponds to an essentially maximum jaw opening, and when the support units are deformed, as a result of increased interjaw compressive forces applied during an epileptic seizure, the separation between the two ribs is reduced. The protection device is arranged such that support units 20 and 25 have bilateral symmetry about plane I-I coincidental with anteriorly located arcuate members 12 and 13 (FIG. 2), which are formed within ribs 15 and 17, respectively, to prevent damage to the frenulum of the upper or lower lip when the protection device is inserted within the oral cavity, as will be described hereinafter. Since the support units are disposed at a posterior end of protection device 10, a centrally located opening 8 is formed between the two support units and between the two ribs, through which air is inhaled into the lungs and through which vomit may be ejected from the oral cavity.

Support units 20 and 25 are provided with a plurality of longitudinally disposed compressible fins 28, which allow a support unit to change shape following an application of increased interjaw compressive forces, yet are sufficiently elastic so as to return the temporarily deformed support unit to its original dimensions following the relaxation of the masticatory muscles. The ribs and the fins are produced from different materials, such that the yield strength and stiffness of the ribs are significantly greater than that of the fins. Accordingly, the protection device is advantageously adapted to both bear the stress associated with increased interjaw compressive forces by means of the relatively stiff ribs and to follow the movement of the jaws by means of the elastically compressible fins without any discomfiture at the cessation of the epileptic seizure.

As shown further in FIGS. 2 and 3, each fin comprises a first portion 28a proximate to rib 15, a second portion 28b proximate to rib 17, and a central arcuate portion 31 connecting first portion 28a and second portion 28b. First portion 28a and second portion 28b are symmetrical about plane II-II, which passes through junction 33 and separates a support unit into two separate sections. First portion 28a is a planar element, and all first portions of a given support unit are mutually parallel. Similarly second portion 28b is a planar element, and all second portions of a given support unit are mutually parallel. Equal spacing is provided between each adjacent first portion 28a and between each adjacent second portion 28b. The length of each first and second portion progressively decreases from the most anteriorly disposed fin to the most posteriorly disposed fin, so that rib 15 is inclined at a predetermined angle with respect to rib 17. This predetermined angle is essentially equal to, and slightly less than, the maximum jaw angular opening, which allows for temporary deformation of a support unit during an uncontrolled occlusal biting motion without any risk of being dislodged from between a corresponding set of upper and lower posterior teeth as the jaws are separated to their fullest extent.

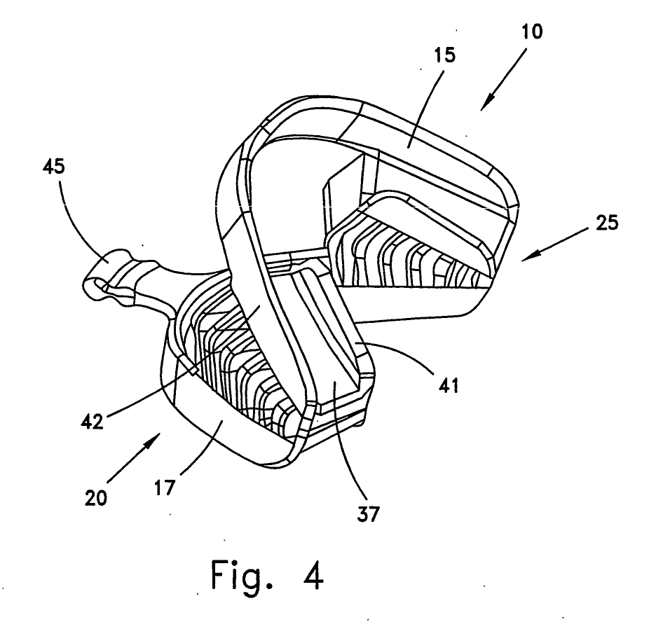

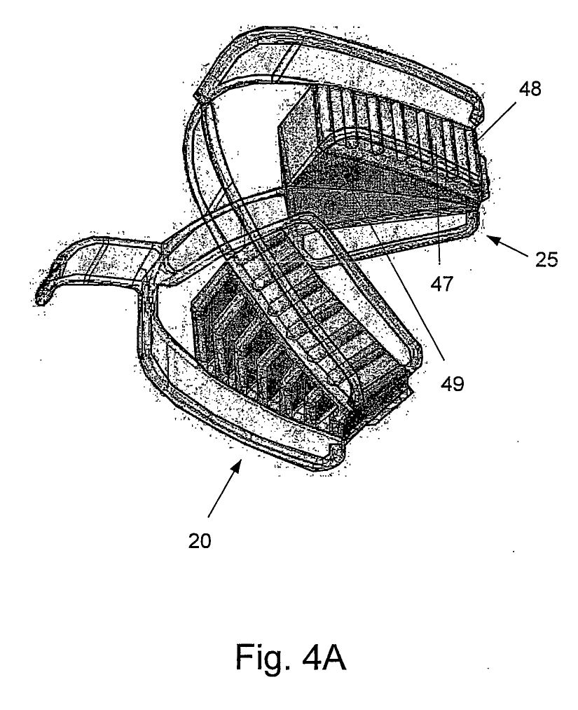

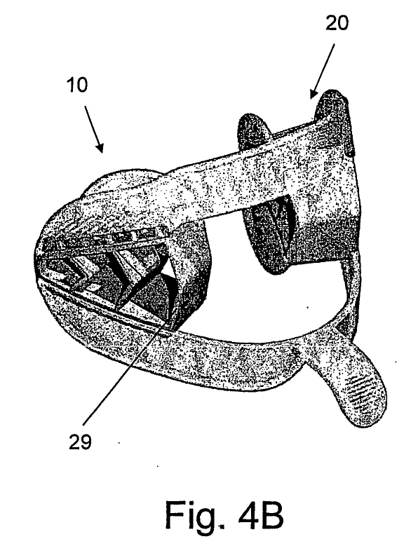

FIG. 4 is a perspective view of protection device 10 at an angle which illustrates a tooth retention surface 37 which is adapted to receive and support a corresponding set of posterior teeth therein during an epileptic seizure. Each tooth retention surface 37 is planar and abuts the same longitudinal end of each fin, whether a first or second portion, extending the entire length of a corresponding support unit. The borders of the illustrated tooth retention surface 37 are defined by wall 41 which longitudinally protrudes from the lingual side thereof and by a portion 42 of rib 15 which longitudinally protrudes from the buccal side thereof

FIG. 4A illustrates another tooth receiving means. Posterior teeth are received on, and supported by, tooth retention surface 47, from which protrude a plurality of curved protrusions 48. Each protrusion 48, which is adapted to retain teeth on surface 47 during jaw movement, extends the width of tooth retention surface 47 and is equally spaced from one another. As shown, each support unit 20 and 25 is provided with a lingual solid wall 49, to provide added rigidity to each support unit.

As shown in FIG. 4B, each support unit 10 and 20 may be configured with as few as three fins 29. Each fin may be chevron shaped as illustrated, or any other suitable shape. If so desired, the ribs and fins may be made of the same material, which is suitable for bearing the stress associated with increased interjaw compressive forces and for following the movement of the jaws by means of the elastically compressible.

Operationally, a subject suffering from epilepsy, who is trained to sense the manifestation of an aura, inserts protection device 10 into his mouth at an appropriate moment by means of handle 45 integrally formed with rib 17, longitudinally inwards from arcuate member 13 (FIG. 2). Handle 45 may be flat as in FIG. 1, circular as in FIG. 2, flared as in FIG. 3, or in any other convenient arrangement. Since ribs 15 and 17 are symmetrical and support units 20 and 25 have bilateral symmetry, the subject may insert the protection device into his mouth in any orientation, that is, the protection device is equally effective if rib 15 is positioned opposite the top teeth or the bottom teeth. The subject inwardly inserts the protection device until each set of posterior teeth, e.g. the set of teeth from the first premolar to the first molar, contacts a corresponding teeth retention surface and one of the ribs contacts the upper gum and the other rib contacts the lower gum. It will be appreciated that one of the advantages of the present invention is the short insertion time, within approximately 5 seconds, required in order to position the protection device in working position within the mouth. Accordingly, due to the configuration of the protection device, arcuate members 12 and 13 are centrally located on the gums and are located longitudinally inwards from the frenulum of a corresponding lip when the protection device is inserted within the oral cavity. However, if some teeth of the subject are misshaped and some of the posterior teeth are therefore not retained by borders 41 and 42, the subject may laterally reposition the protection device, if time permits, so that all of said set of posterior teeth contact a corresponding teeth retention surface. The protection device will effectively function in accordance with the present invention even if some or all of the posterior teeth are missing because of its active adaptability to occluding surfaces, whether they are teeth or gums.

As previously stated, rib 15 is inclined with respect to rib 17 at an angle substantially equal to the maximum jaw angular opening. Accordingly, ribs 15 and 17 follow the movement of the jaws while the posterior teeth continue to contact the corresponding teeth retention surface. When an uncontrollable longitudinal occlusal biting motion takes place, teeth retention surface 37 is depressed and the fins of a support unit are momentarily deformed in response to the magnitude and direction of the interjaw compressive force. Upon conclusion of the biting motion, the masticatory muscles relax and the support units revert to their original configuration at which the inter-rib angle is essentially equal to the maximum jaw angular opening. Failure of a support unit due an excessive stress concentration is precluded since the fins compress and the ribs distribute the interjaw compressive forces. Similarly, when an uncontrollable transversal biting motion takes place, the fins of a support unit are momentarily deformed in response to a transversal interjaw compressive force. The posterior teeth are retained by one of the borders 41 or 42 and remain in contact with teeth retention surface 37. Since the posterior teeth remain within borders 41 and 42 during an epileptic seizure and upper posterior teeth are continuously separated from corresponding lower posterior teeth, a given set of posterior teeth cannot inflict injury to the tongue, inner cheeks or other teeth of the subject. The tongue therefore needs not to be restrained as in prior art protection devices and is free to facilitate swallowing or vomit ejection. During tongue convulsions or thrusts, the protection device cannot be ejected from the oral cavity due to the pressure exerted by the posterior teeth on the support units and by the friction exerted by the inner lips and gums on arcuate members 12 and 13 (FIG. 2).

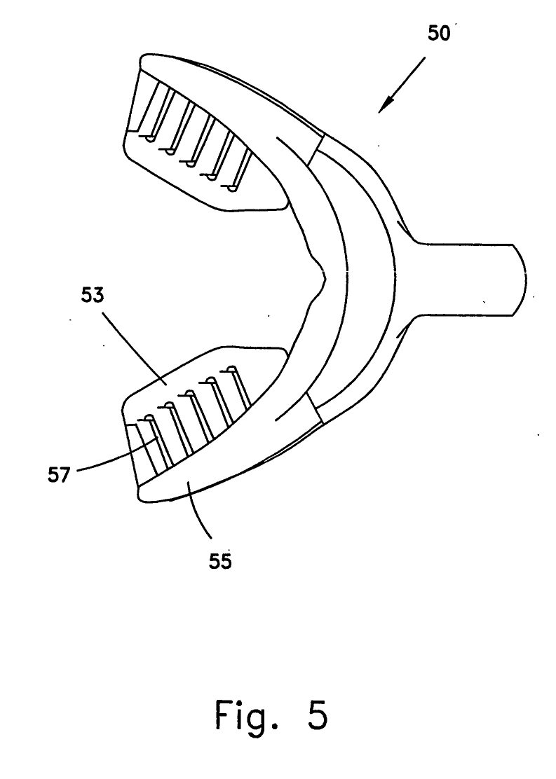

FIG. 5 illustrates a second preferred embodiment of the present invention and is indicated generally by numeral 50. Protection device 50, which is similar in shape and function as protection device 10 of FIG. 1, is advantageously provided with a solid lingual wall 53 and a solid buccal wall 55 for each support unit. A plurality of chambers are thereby defined by walls 53 and 55 and by two adjacent fins 57. Different types of therapeutic material may be stored in each of the chambers, such as medicine, scent or taste additives, temperature or pressure induced material to be released to the oral cavity.

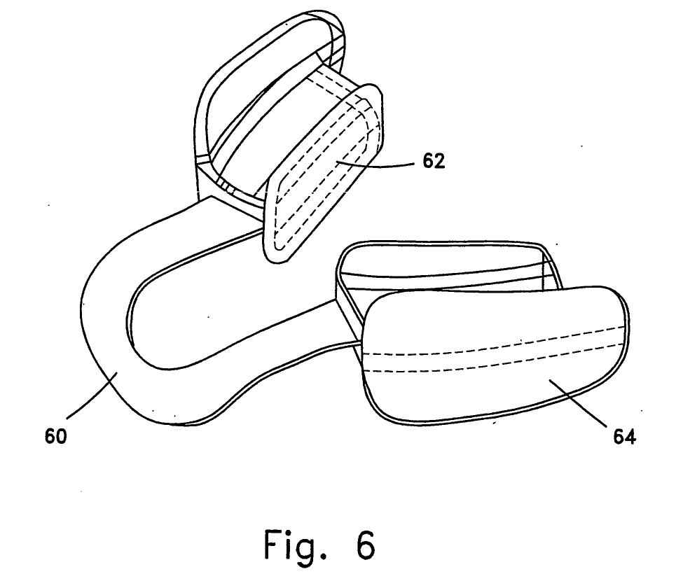

FIG. 6 illustrates a third embodiment of the present invention in which one U-shaped rib 60 is attached at its posterior ends to support units 62 and 64, functionally similar to the support units of FIG. 1.

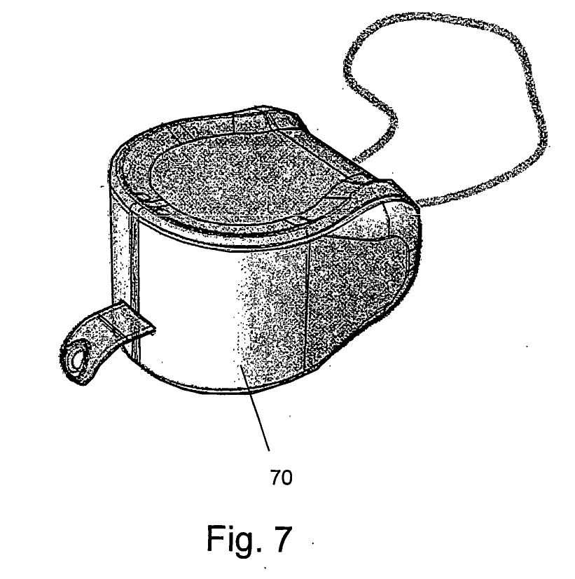

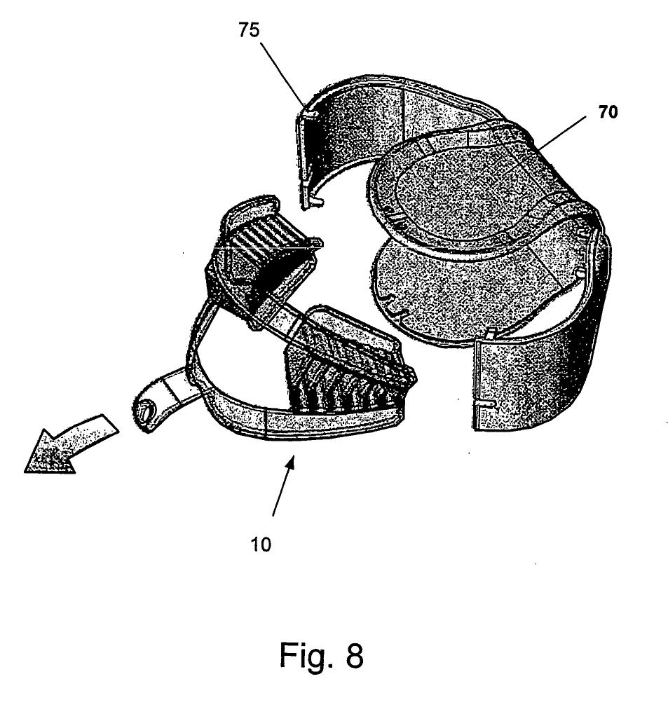

FIGS. 7 and 8 illustrate container 70 which is adapted for holding protection device 10. The dimensions of protection device 10 may be modified to conform to those of any subject including children, and accordingly the dimensions of protection device may also be modified. The protection device may be manufactured from an inexpensive material so that it may be disposable and be adapted for one-time use. Alternatively, the protection device may be reusable. The protection device is removed from the container in pop-up or pull-out fashion by simply pulling the handle of the protection device while grasping container 70. Pins 75 are then dislodged from their points of fixation within the walls of the container, thereby breaking the container and rendering it unusable. Alternatively, the container may be configured such that the pins will break upon removal of the protection device from said container. The container may be provided with a retaining means, such as a loop, grip or string, by which the container may be retained in an accessible location, such as around the neck, so that it may be quickly inserted into the oral cavity of a subject upon manifestation of an aura.

The apparatus of the present invention as described hereinbefore can be advantageously adapted to other applications, in addition to those relating to epileptic seizures. When a protection device is inserted into the mouth of a sport practitioner, the practitioner is protected from externally afflicted injury, such as during a collision, to the teeth, inner cheeks or tongue when running, jumping or any other strenuous activity. Devices of the prior art were needed to be physically held in place to prevent their ejection from the mouth of the sport practitioner when inhaling and exhaling deep breaths during strenuous activity. In contrast, the device of the present invention may remain in the mouth of the sport practitioner throughout his sport activity.

The device of the present invention may also be adapted for suppressing upper airway resistance syndrome, sleep apnea syndrome and/or snoring by separating the upper jaw from the lower jaw, thereby precluding contact between the tongue and palate and minimizing the possibility of oropharyngeal obstruction.

The device of the present invention may also be used as a means for immobilizing the temporomandibular joint following jaw surgery to prevent permanent scarring. Similarly it may be used for physiotherapy and physical therapy, such as in exercise therapy, e.g. therapeutic movement of the jaws without tooth contact, and muscle strengthening exercises, assisting jaws with fibrotic joints to open to a larger extent or to provide relief to patients suffering from myofascial pain disorder (MPD), which is a condition in which the masticatory muscles are tensed in a painful way causing a disturbance to the oral functions and for which immediate relief may be obtained by opening the mouth of the patient, thereby providing muscle relaxation.

The device of the present invention may also be used to deliver therapeutic and/or edible material to a subject. The material may be stored in reservoirs bored or otherwise formed in a support unit. The material is delivered to a subject by means selected from the group of the pressure applied to the deformable member by the jaws, temperature activated means, moisture activated means, timed release means and control means which may comprise at least one electronic component and circuitry. Control means or measuring means may be provided in the reservoirs, which control and/or monitor electric currents in the mouth and cheeks, as well as saliva secretion. The material may be immediately deliverable upon activation of the delivery means, such as within 5 seconds. The material may be a pharmaceutically active agent or a scent or taste additive to the oral cavity. The material may be in the form selected from the group of liquid, aerosol, powder, gas, and encapsulated form.

While some embodiments of the invention have been described by way of illustration, it will be apparent that the invention can be carried into practice with many modifications, variations and adaptations, and with the use of numerous equivalents or alternative solutions that are within the scope of persons skilled in the art, without departing from the spirit of the invention or exceeding the scope of the claims.

1. A device for separating between the upper and lower jaws, comprising at least one substantially U-shaped rib and a deformable member associated therewith such that said member gradually changes its shape when a pressure is applied on said at least one rib by the jaws.

2. Device according to claim 1, wherein the device is a protection device which prevents a self-inflicting or externally inflicted injury.

3. Device according to claim 1, wherein the device is a device for suppressing upper airway resistance syndrome, sleep apnea syndrome or snoring.

4. Device according to claim 1, comprising two substantially U-shaped ribs, connected at at least one end to one another, and a deformable member positioned between them such that said member gradually changes its shape when a pressure is applied on said ribs by the jaws.

5. Device according to claim 4, comprising:
a) two U-shaped ribs having a lingual and buccal side, each of said ribs having a curvature corresponding to the curvature of a dental arch and subtending a majority of its length;
b) two support units attached at each posterior end of a pair of said ribs on the lingual side thereof, such that said two support units have bilateral symmetry about a plane coincidental with an anteriorly located central portion of each of said ribs and that each of said ribs is separated one from the other, at a given point along a rib, by a separation substantially corresponding to an essentially maximum jaw angular opening when said support units are not compressed; and
c) an upper and a lower tooth receiving means for each support unit, each of said tooth receiving means extending the entire length of a corresponding support unit and adapted to retain posterior teeth therein, borders of each of said tooth receiving means being defined by a wall of a support unit longitudinally protruding from a lingual side of a corresponding tooth receiving means and a portion of a rib longitudinally protruding from the buccal side thereof.

6. Device according to claim 5, wherein each support unit comprises a plurality of longitudinally disposed fins which are compressible upon application of interjaw forces and which can return to their original dimensions following the relaxation of the jaw muscles.

7. Device according to claim 5, wherein each support unit is provided with solid lingual walls.

8. Device according to claim 5, wherein each support unit is enclosed by solid lingual and buccal walls, a plurality of chambers being defined by said solid walls and by two adjacent fins for the insertion therein of therapeutic material.

9. Device according to claim 6, wherein each fin comprises a first portion proximate to a first rib, a second portion proximate to a second rib, and a central arcuate portion connecting said first portion and said second portion, said first portion and second portions being symmetrical about a plane which passes through a junction connecting said first and second ribs and which separates a support unit into two separate sections.

10. Device according to claim 9, wherein the first and second portions are planar elements, all first portions of a given support unit being mutually parallel and all second portions of a given support unit being mutually parallel.

11. Device according to claim 10, wherein the length of each first and second portion preferably progressively decreases from the most anteriorly disposed fin to the most posteriorly disposed fin whereby the first rib is inclined at a predetermined angle with respect to the second rib.

12. Device according to claim 11, wherein the predetermined angle is essentially equal to the maximum jaw angular opening.

13. Device according to claim 6, wherein the tooth receiving means is a planar surface which abuts the same longitudinal end of each fin of a support unit.

14. Device according to claim 7, wherein the tooth receiving means is the plurality of chambers.

15. Protection device according to claim 1, wherein the central anteriorly disposed portion of each rib is an arcuate member for retaining incisor teeth on the buccal side thereof.

16. Protection device according to claim 15, further comprising a handle integrally formed with a rib, longitudinally inwards from the corresponding arcuate member.

17. Device according to claim 1, further comprises at least one reservoir suitable for housing a beneficial or edible material.

18. Device according to claim 17, wherein the beneficial or edible material is delivered to a subject by means selected from the group of the pressure applied to the deformable member by the jaws, temperature activated means, moisture activated means, timed release means and control means.

19. Device according to claim 18, wherein the control means is at least one electronic component and circuitry.

20. Device according to claim 18, wherein the at least one reservoir is additionally suitable for housing a measuring or control means.

21. Device according to claim 19, wherein the measuring or control means measures or controls electric current and/or saliva secretion.

22. Device according to claim 18, wherein the beneficial or edible material is essentially immediately deliverable upon activation of the delivery means.

23. Device according to claim 17, wherein the beneficial or edible material is a pharmaceutically active agent.

24. Device according to claim 17, wherein the beneficial or edible material is a scent or taste additive to the oral cavity.

25. Device according to claim 17, wherein the beneficial or edible material is in the form selected from the group of liquid, aerosol, powder, gas, and encapsulated form.

26. Device according to claim 17, wherein each reservoir is a recess formed in the deformable member.

27. Device according to claim 5, wherein the central anteriorly disposed portion of each rib is placeable on a corresponding gum when the device is inserted within the oral cavity.

28. Device according to claim 27, wherein the central anteriorly disposed portion of each rib is an arcuate member which is shaped so as to prevent damage to the frenulum of a corresponding lip.

29. Device according to claim 5, wherein the device further comprises a handle integrally formed with a rib, longitudinally inwards from a corresponding central anteriorly disposed portion.

30. Device according to claim 5, wherein the device is formed with a centrally and anteriorly located airway for the release of vomit from the oral cavity.

31. Device according to claim 30, wherein the airway is formed between two opposing ribs and two opposing deformable members.

32. Device according to claim 1, wherein the at least one rib and the deformable member are produced from different materials, the yield strength of the at least one rib being significantly greater than that of the deformable member.

33. Device according to claim 1, wherein the at least one rib and the deformable member are produced from the same material.

34. Device according to claim 1, wherein the material of the deformable member hardens after use.

35. Device according to claim 1, wherein the device is disposable and for one-time use.

36. Device according to claim 1, wherein the device is reusable.

37. Device according to claim 1, wherein the device is packageable in a container which breaks upon removal of the device from said container.

38. Device according to claim 37, wherein the container is provided with a retaining means for retaining the container in an accessible location.

39. Device according to claim 5, wherein each support unit is configured in such a way and produced from a suitable material so as to follow the movement of the jaws.

40. A method for delivering material to a subject, comprising:
a) providing a device with two substantially U-shaped ribs, connected at at least one end to one another, and a deformable member positioned between them formed with at least one reservoir suitable for housing a beneficial or edible material;
b) filling each of said at least one reservoir with a beneficial or edible material;
c) inserting said device into the oral cavity of said subject such that said deformable member separates an upper set of posterior teeth from a corresponding lower set of posterior teeth; and
d) allowing said beneficial or edible material to be delivered to said subject.

41. Method according to claim 40, wherein the beneficial or edible material is delivered to the subject upon application of pressure onto said deformable member by the jaws.

42. Method according to claim 40, wherein the beneficial or edible material is delivered to the subject when the temperature within the oral cavity of the subject is greater than a predetermined value.

43. Method according to claim 40, wherein the beneficial or edible material is delivered to the subject when the moisture level within the oral cavity of the subject is greater than a predetermined value.

44. Method according to claim 40, wherein the beneficial or edible material is delivered to the subject after a predetermined time following insertion of the device within the oral cavity.

45. Method according to claim 40, wherein the beneficial or edible material is delivered to the subject upon activation of control circuitry.
